(19) United States
(12) Patent Application Publication
(10) Pub. No.: US 2022/0273531 A1
(43) Pub. Date: Sep. 1, 2022

(54) COLOURING EFFECT PIGMENTS AND PRODUCTION THEREOF

(71) Applicant: Henkel AG & Co. KGaA, Duesseldorf (DE)

(72) Inventor: Torsten LECHNER, Langenfeld (DE)

(73) Assignee: Henkel AG & Co. KGaA, Duesseldorf (DE)

(21) Appl. No.: 17/628,474

(22) PCT Filed: Jul. 6, 2020
(86) PCT No.: PCT/EP2020/068961

§ 371 Date: Jan. 19, 2022

(30) Foreign Application Priority Data
Jul. 19, 2019 (DE) 10 2019 210 687.0

(51) Int. Cl. A61K 8/02; A61K 8/26; A61K 8/25; A61Q 5/10; C09C 1/00

(52) U.S. Cl. CPC A61K 8/0266; A61K 8/26; A61K 8/25; A61Q 5/10; C09C 1/0075; A61K 2800/621; A61K 2800/651; A61K 2800/436; C09C 2200/1054; C01P 2004/20; C09C 2220/106

(57) ABSTRACT

The application describes an effect pigment comprising a) a substrate platelet and b) a coating. The coating has at least one layer that comprises (i) a metal oxide and/or metal oxide hydrate and

(ii) a coloring compound from the group of pigments.

A process for producing the colored effect pigments is also described.

### CROSS-REFERENCE TO RELATED APPLICATION

This application is a U.S. National-Stage entry under 35 U.S.C. § 371 based on International Application No. PCT/EP2020/068961, filed Jul. 6, 2020, which was published under PCT Article 21(2) and which claims priority to German Application No. 102019210687.0, filed Jul. 19, 2019, which are all hereby incorporated in their entirety by reference.

### TECHNICAL FIELD

The present application relates to effect pigments comprising a substrate platelet and a coating, the coating having at least one layer comprising a metal oxide and/or metal oxide hydrate. The application further describes a process to produce the effect pigments.

### BACKGROUND

The change in shape and color of keratin fibers, especially hair, is a key area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between about 5 and about 20 washes.

The use of color pigments is known for short-term color changes on the hair and/or skin. Color pigments are understood to be insoluble, coloring substances. These are present undissolved in the dye formulation in the form of small particles and are only deposited from the outside on the hair fibers and/or the skin surface. Therefore, they can usually be removed without residue by a few washes with surfactant-comprising cleaning agents. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when the combination of a pigment, an organic silicon compound, a film-forming polymer and a solvent is used on hair, it is possible to create colorations that are particularly resistant to shampooing.

Metallic luster pigments or metallic effect pigments are widely used in many fields of technology. They are used, for example, to color coatings, printing inks, inks, plastics, glasses, ceramic products and preparations for decorative cosmetics such as nail polish. They are exemplified by their attractive angle-dependent color impression (goniochromism) and their metallic-looking luster.

Hair with a metallic finish or metallic highlights are in trend. The metallic tone makes the hair look thicker and shinier.

### BRIEF SUMMARY

This disclosure provides an effect pigment comprising a) a substrate platelet and b) a coating, wherein the b) coating comprises at least one layer which comprises (i) a metal oxide and/or metal oxide hydrate; and (ii) a coloring compound chosen from the group of pigments.

This disclosure also provides a process for the preparation of an effect pigment comprising a) a substrate platelet and b) a coating, wherein the coating comprises at least one layer which comprises (i) a metal oxide and/or metal oxide hydrate and (ii) a coloring compound chosen from pigments, said process comprising the steps of: (α) suspending the substrate platelet in an organic or aqueous solvent, and (β) coating the substrate platelet suspended in step (α) with the at least one layer using a sol-gel process.

This disclosure further provides a process for the preparation of an effect pigment comprising: suspending Al platelets having a thickness of from about 20 to about 30 nm in isopropanol; adding tetraethoxysilane and a pigment to the Al platelets suspended in the isopropanol to form a mixture; heating the mixture; and filtering the mixture to provide the effect pigment comprising a pigmented silica layer disposed thereon.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

There is a need to provide effect pigments, especially for hair dyeing, which on the one hand have high wash and rub fastness and on the other hand do not negatively affect hair properties such as manageability and feel. For this purpose, it would be desirable if the effect pigments used had a high covering power and could be applied to the hair in thin layers. It would also be desirable if the effect pigments could be used to dye a material to be colored, hair, with a wide range of metallic colors.

The effect pigments should be particularly suitable for dyeing systems that do not require the use of oxidizing agents and/or oxidation dye precursors.

Surprisingly, it has now been found that the tasks can be excellently solved by an effect pigment comprising a) a substrate platelet and b) a coating, wherein the coating comprises at least one layer which is

(i) a metal oxide and/or metal oxide hydrate and
(ii) a coloring compound from the group of pigments.

The effect pigment has a substrate platelet.

The substrate wafer preferably has an average thickness of at most about 50 nm, preferably less than about 30 nm, particularly preferably at most about 25 nm, for example at most about 20 nm. The average thickness of the substrate platelets is at least about 1 nm, preferably at least about 2.5 nm, particularly preferably at least about 5 nm, for example at least about 10 nm. Preferred ranges for substrate wafer thickness are about 2.5 to about 50 nm, about 5 to about 50 nm, about 10 to about 50 nm; about 2.5 to about 30 nm, about 5 to about 30 nm, about 10 to about 30 nm; about 2.5 to about 25 nm, about 5 to about 25 nm, about 10 to about 25 nm, about 2.5 to about 20 nm, about 5 to about 20 nm, and about 10 to about 20 nm. Preferably, each substrate plate has a thickness that is as uniform as possible.

The substrate plate is preferably monolithic. Monolithic in this context means comprising a single self-included unit without fractures, stratifications or inclusions, although microstructural changes may occur within the substrate platelet. The substrate platelet is preferably homogeneous in structure, i.e., no concentration gradient occurs within the platelet. In particular, the substrate platelet is not layered and does not have particles or particulates distributed therein.

The size of the substrate platelet can be tailored to the specific application, for example the desired effect on a keratinous material. Typically, the substrate platelets have an average largest diameter of about 2 to about 200 μm, especially about 5 to about 100 μm.

In a preferred embodiment, the shape factor (aspect ratio), expressed by the ratio of the average size to the average thickness, is at least about 80, preferably at least about 200, more preferably at least about 500, particularly preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for about 3 minutes.

The substrate platelet can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-) precious stones, and plastics. Preferably, the substrate plates are constructed of a metal or alloy.

Any metal suitable for effect pigments can be used. Such metals include iron and steel, as well as all air- and water-resistant (semi)metals such as platinum, tin, zinc, chromium, molybdenum and silicon, as well as their alloys such as aluminum bronzes and brass. Preferred metals are aluminum, copper, silver and gold. Preferred substrate platelets include aluminum platelets and brass platelets, with aluminum substrate platelets being particularly preferred. Substrate plates made of aluminum can be produced, among other things, by punching out of aluminum foil or according to common milling and atomization techniques. For example, aluminum flakes are available from the Hall process, a wet milling process.

Other metal flakes, for example of bronze, can be obtained in a dry grinding process such as the Hametag process.

The substrate plates can have different shapes. For example, lamellar or lenticular metal platelets or so-called vacuum metallized pigments (VMP) can be used as substrate platelets. Lamellar substrate platelets are exemplified by an irregularly structured edge and are also referred to as “cornflakes” due to their appearance. Lenticular substrate flakes have a regular round edge and are also known as “silverdollars” because of their appearance.

The metal or metal alloy substrate plates can be passivated, for example by anodizing (oxide layer) or chromating.

A coating can change the surface properties and/or optical properties of the effect pigment and increase the mechanical and chemical load-bearing capacity of the effect pigments. For example, only the upper and/or lower side of the substrate wafer may be coated, with the side surfaces being recessed. Preferably, the entire surface of the optionally passivated substrate platelets, including the side surfaces, is covered by the layer. The substrate platelets are preferably completely encased by the coating.

The coating may include one or more layers. In a preferred embodiment, the coating has only layer A. In a likewise preferred embodiment, the coating has a total of at least two, preferably two or three, layers. It may be preferred to have the coating have two layers A and B, with layer B being different from layer A. Preferably, layer A is located between layer B and the surface of the substrate plate. In yet another preferred embodiment, the coating has three layers A, B and C. In this embodiment, layer A is located between layer B and the surface of the substrate wafer and layer C is located on top of layer B, which is different from the layer B below.

Suitable materials for layers A and, if necessary, B and C are all substances that can be permanently applied to the substrate platelets. The materials should preferably be applicable in film form. Preferably, the entire surface of the optionally passivated substrate platelets, including the side surfaces, is enveloped by layer A or by layers A and B or by layers A, B and C.

It is preferred that the metal oxide and/or metal oxide hydrate (i) is selected from the group of silicon (di)oxide, silicon oxide hydrate, aluminum oxide, aluminum oxide hydrate, boron oxide, germanium oxide, manganese oxide, magnesium oxide, iron oxide, cobalt oxide, chromium oxide, titanium dioxide, vanadium oxide, zirconium oxide, tin oxide, zinc oxide and mixtures thereof.

Layer A preferably has at least one low refractive index metal oxide and/or metal oxide hydrate. Preferably, layer A comprises at least about 95% by weight of low refractive index metal oxide (hydrate). Low refractive index materials have a refractive index of about 1.8 or less, preferably about 1.6 or less.

Low refractive index metal oxides suitable for Layer A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. Layer A preferably has a thickness of about 1 to about 100 nm, particularly preferably about 5 to about 50 nm, especially preferably about 5 to about 20 nm.

Layer B, if present, is different from Layer A and may contain at least one highly refractive metal oxide. Highly refractive materials have a refractive index of at least about 1.9, preferably at least about 2.0, and more preferably at least about 2.4. Preferably, layer B comprises at least about 95 wt. %, more preferably at least about 99 wt. %, of high refractive index metal oxide(s).

If the layer B comprises a (highly refractive) metal oxide, it preferably has a thickness of at least about 50 nm. Preferably, the thickness of layer B is no more than about 400 nm, more preferably no more than about 300 nm.

Highly refractive metal oxides suitable for layer B are, for example, selectively light-absorbing (i.e., colored) metal oxides, such as iron(III) oxide (α- and γ-Fe2O3, red), cobalt(II) oxide (blue), chromium(III) oxide (green), titanium(III) oxide (blue, usually present in admixture with titanium oxynitrides and titanium nitrides), and vanadium(V) oxide (orange), as well as mixtures thereof. Colorless high-index oxides such as titanium dioxide and/or zirconium oxide are also suitable.

Layer B can contain a selectively absorbing dye in addition to a highly refractive metal oxide, preferably about 0.001 to about 5% by weight, particularly preferably about 0.01 to about 1% by weight, in each case based on the total amount of layer B. Suitable dyes are organic and inorganic dyes that can be stably incorporated into a metal oxide coating. Dyes in the sense of the present disclosure have a solubility in water (760 mmHg) at 25° C. of more than about 0.5 g/L and are therefore not to be regarded as pigments.

Alternatively, to a metal oxide, layer B may comprise a metal particle carrier layer with metal particles deposited on the surface of the metal particle carrier layer. In a preferred embodiment, the metal particles directly cover a portion of the metal particle carrier layer. In this embodiment, the effect pigment has areas in which there are no metal particles, i.e., areas which are not covered with the metal particles.

The metal particle carrier layer comprises a metal layer and/or a metal oxide layer.

If the metal particle carrier layer comprises a metal layer and a metal oxide layer, the arrangement of these layers is not limited.

It is preferred that the metal particle support layer at least comprises a metal layer. It is further preferred that the metal layer comprises an element selected from tin (Sn), palladium (Pd), platinum (Pt) and gold (Au).

The metal layer can be formed, for example, by adding alkali to a metal salt solution comprising the metal.

If the metal particle carrier layer comprises a metal oxide layer, this preferably does not comprise silicon dioxide. The metal oxide layer preferably comprises an oxide of at least one element selected from the group of Mg (magnesium), Sn (tin), Zn (zinc), Co (cobalt), Ni (nickel), Fe (iron), Zr (zirconium), Ti (titanium) and Ce (cerium). Particularly preferably, the metal particle support layer iii) in the form of a metal oxide layer comprises a metal oxide of Sn, Zn, Ti and Ce.

The metal particle support layer in the form of a metal oxide layer can be produced, for example, by hydrolysis of an alkoxide of a metal forming the metal of the metal oxide in a sol-gel process.

The thickness of the metal particle support layer is preferably not more thaw about 30 nm.

The metal particles may comprise at least one element selected from the group of aluminum (Al), titanium (Ti), chromium (Cr), iron (Fe), cobalt (Co), nickel (Ni), copper (Cu), zinc (Zn), ruthenium (Ru), rhodium (Rh), palladium (Pd), silver (Ag), tin (Sn), platinum (Pt), gold (Au), and alloys thereof. It is particularly preferred that the metal particles comprise at least one element selected from copper (Cu), nickel (Ni) and sifter (Ag).

The average particle diameter of the metal particles is preferably not more than about 50 nm, more preferably not more than about 30 nm. The distance between the metal particles is preferably not more than about 10 nm.

Suitable methods for forming the metal particles include vacuum evaporation, sputtering, chemical vapor deposition (CVD), electroless plating, or the like. Of these processes, electroless plating is particularly preferred.

According to a preferred embodiment, the effect pigments have a further layer C, comprising a metal oxide (hydrate), which is different from the layer B underneath. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The layer C preferably has a thickness of about 10 to about 500 nm, more preferably about 50 to about 300 nm.

The coating of the effect pigment has at least one layer which, in addition to the metal oxide and/or the metal oxide hydrate, further comprises a color-imparting compound from the group of pigments.

The at least one layer comprising (i) a metal oxide and/or metal oxide hydrate and (ii) a coloring compound selected from the group of pigments may be layer A, B and/or C. In the case where the coating has only layer A, layer A also comprises the colorant compound from the group of pigments.

In the case where the coating of the effect pigment has two layers A and B and each comprises a metal oxide, both layers A and B or only one of the two layers may comprise the color-imparting compound from the group of pigments. Preferably, layer A comprises the colorant compound from the group of pigments.

In the case where the coating has layers A, B, and C and each comprises a metal oxide (hydrate), each of layers A, B, and C may contain a color-imparting compound selected from the group of pigments. Alternatively, in this embodiment, two of the three layers may contain the coloring compound from the group of pigments. Accordingly, the colorant compound can be from the group of pigments in layer A and B, in layer A and C, or in layer B and C. Similarly, only one of the three layers may comprise a colorant compound from the group of pigments. Accordingly, the coloring compound can be from the group of pigments in layer A, B or C. In a particularly preferred embodiment of an effect pigment comprising a coating with layers A, B and C, the color-imparting compound is from the group of pigments in layer A and/or C.

In the case where the coating has layers A, B and C, layers A and C comprise a metal oxide (hydrate) and layer B comprises a metal layer with metal particles deposited thereon, each of layers A and C may contain a color-imparting compound selected from the group of pigments. Alternatively, in this embodiment, only one of the layers A and C may contain the colorant compound selected from the group of pigments.

In a particularly preferred embodiment of an effect pigment comprising a coating with layers A, B and C, the color-imparting compound is from the group of pigments in layer A and/or C.

It is particularly preferred that the effect pigment comprises a substrate platelet of aluminum and a layer A comprising silica. If the effect pigment based on a substrate platelet has a layer A and a layer C, it is preferred that the effect pigment has a substrate platelet of aluminum and layers A and C comprising silica.

Pigments within the meaning of the present disclosure are coloring compounds which have a solubility in water at about 25° C. of less than about 0.5 g/L, preferably less than about 0.1 g/L, even more preferably less than about 0.05 g/L. Water solubility can be determined, for example, by the method described below: about 0.5 g of the pigment are weighed in a beaker. A stir-fish is added. Then one liter of distilled water is added. This mixture is heated to about 25° C. for one hour while stirring on a magnetic stirrer. If undissolved components of the pigment are still visible in the mixture after this period, the solubility of the pigment is below about 0.5 g/L. If the pigment-water mixture cannot be assessed visually due to the high intensity of the finely dispersed pigment, the mixture is filtered. If a proportion of undissolved pigments remains on the filter paper, the solubility of the pigment is below about 0.5 g/L.

Suitable color pigments can be of inorganic and/or organic origin.

In a preferred embodiment, the effect pigment comprises at least one color-imparting compound selected from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

Also preferred mica-based pigments are synthetically produced mica platelets coated with metal oxide, based on synthetic fluorophlogopite (INCI: Synthetic Fluorphlogopite). The synthetic fluorophlogopite platelets are coated with, for example, tin oxide, iron oxide(s) and/or titanium dioxide. The metal oxide layers may further comprise pigments such as ferric hexacyanidoferrate(II/III) or carmine red. Such mica pigments are available, for example, under the name SYNCRYSTAL from Eckart.

Accordingly, a preferred effect pigment is exemplified wherein it comprises at least one coloring compound from the group of pigments selected from the group of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

In a further preferred embodiment, the effect pigment is exemplified wherein it comprises at least one coloring compound from the group of pigments selected from mica- or mica-based pigments which are reacted with one or more metal oxides from the group comprising titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Other suitable pigments are based on metal oxide-coated platelet-shaped borosilicates. These are coated with tin oxide, iron oxide(s), silicon dioxide and/or titanium dioxide, for example. Such borosilicate-based pigments are available, for example, under the name MIRAGE from Eckart or Reflecks from BASF SE.

Examples of particularly suitable pigments are commercially available under the trade names Rona®, Colorona®, Xirona®, Dichrona® and Timiron® from Merck, Ariabel® and Unipure® from Sensient, Prestige® from Eckart Cosmetic Colors, Flamenco®, Cellini®, Cloisonné®, Duocrome®, Gemtone®, Timica®, MultiReflections, Chione from BASF SE and Sunshine® from Sunstar.

Very particularly preferred pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Colorona SynCopper, Merck, Synthetic Fluorphlogopite (and) Iron Oxides
Colorona SynBronze, Merck, Synthetic Fluorphlogopite (and) iron Oxides

Further particularly preferred pigments with the trade name Xirona® are, for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin

Xirona Le Rouge, Merck, Iron Oxides (and) Silica

In addition, particularly preferred pigments with the trade name Unipure® are, for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In addition, particularly preferred pigments with the trade name Unipure® are, for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the effect pigment may also contain one or more color-imparting compounds from the group of organic pigments.

The organic pigments are correspondingly insoluble organic dyes or colorants which may be selected, for example, from the group of nitroso, nitro-azo, xanthene, anthraquinone, isoindolinone, isoindoline, quinacridone, perinone, perylene, diketopyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, the effect pigment is exemplified wherein it comprises a coloring compound from the group of organic pigments selected from the group of carmine, quinacridone, phthalocyanine, sorghum, blue pigments with the color index numbers Cl 42090, CI 69800, CI 69825, CI 73000, CI 74100, CI 74160, yellow pigments with the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470 and mixtures thereof.

The organic pigment can also be a color paint. As contemplated herein, the term color lacquer means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

Also, suitable colorant compounds from the group of pigments are inorganic and/or organic pigments modified with a polymer. The polymer modification can, for example, increase the affinity of the pigments to the respective material of the at least one layer.

The particle size of the colorant compound used depends on the layer in which the colorant layer is present. The color-imparting compound preferably has a particle size D₉₀, which is smaller than the layer thickness of the at least one layer. More preferably, the particle size D₉₅ of the coloring compound is smaller than the layer thickness of the at least one layer. Even more preferably, the particle size D₉₉ of the colorant compound is smaller than the layer thickness of the at least one layer. Very preferably, the particle size D₁₀₀ of the coloring compound is smaller than the layer thickness of the at least one layer. The particle size of the coloring compound can be determined using, for example, dynamic light scattering (DLS) or static light scattering (SLS). D₉₀ means that about 90% of the particles of the coloring compound are smaller than the layer thickness of the at least one layer. Accordingly, D₉₅ means that about 95% of the particles of the coloring compound are smaller than the layer thickness of the at least one layer, etc.

The amount of colorant compound from the group of pigments in the layer comprising

(b1) a metal oxide and/or metal oxide hydrate and
(b2) a coloring compound from the group of pigments,
is preferably up to about 5% by weight, based on the total weight of the layer.

Layers A and C serve as corrosion protection as well as chemical and physical stabilization. Particularly preferably, layers A and C contain silicon dioxide or aluminum oxide applied by the sol-gel process.

Accordingly, a further subject matter of the application is a process for preparing an effect pigment comprising a) a substrate platelet and b) a coating, wherein the coating comprises at least one layer that is

(i) a metal oxide and/or metal oxide hydrate and
(ii) comprising a coloring compound selected from the group of pigments, comprising the steps:
(α) suspending the substrate wafer in an organic or aqueous solvent; and
(β) coating the substrate wafer suspended in step (α) with a layer comprising (i) a metal oxide and/or metal oxide hydrate and (ii) a coloring compound selected from the group of pigments, using a sol-gel process.

It is preferred that a metal alkoxide and a colorant compound from the group of pigments are used in the sol-gel process.

It is further preferred that the metal alkoxide used in the sol-gel process is selected from the group of tetramethyl orthosilicate, tetraethyl orthosilicate, tetraisopropyl orthosilicate, and mixtures thereof, with tetraethyl orthosilicate being preferred.

In a preferred embodiment of the manufacturing process, the substrate wafer used in step (α) has already been coated with at least one layer of a metal oxide and/or metal oxide hydrate.

An exemplary manufacturing process comprises dispersing the uncoated substrate platelets or the substrate platelets already coated with layer A or with layers A and B and the colorant compound selected from the group of pigments in a solution of a metal alkoxide such as tetraethyl orthosilicate or aluminum triisopropanolate (usually in a solution of organic solvent or a mixture of organic solvent and water with at least about 50 wt. % organic solvent such as a C1 to C4 alcohol), and adding a weak base or acid to hydrolyze the metal alkoxide, thereby forming a film comprising the metal oxide and the colorant compound selected from the group of pigments on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-comprising layer to the oxide layers by annealing).

Although each of the layers A, B and/or C may contain a mixture of two or more metal oxide(hydrate)s, each of the layers preferably comprises only one metal oxide(hydrate) at a time.

The effect pigments based on coated substrate platelets preferably have a thickness of about 70 to about 500 nm, particularly preferably about 100 to about 400 nm, especially preferably about 150 to about 320 nm, for example about 180 to about 290 nm. The low thickness of the coated substrate platelets is achieved by keeping the thickness of the uncoated substrate platelets low, but also by adjusting the thicknesses of the coatings A and, if present, C to as small a value as possible.

The adhesion and abrasion resistance of effect pigments based on substrate platelets to/in a material, preferably keratinous material, can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-comprising, layer A, B, or C. The outermost layer denotes the layer that is spatially farthest from the substrate platelet. The organic compounds are preferably functional silane compounds that can bind to the metal oxide-comprising layer A, B, or C. These can be either mono- or bifunctional compounds. Examples of bifunctional organic compounds are Methacryloxypropenvltrimethoxysilane, 3-Methacryloxypropyltrimethoxysilane, 3-Acryloxypropyltrimethoxysilane, 2-Acryloxyethyltrimethoxysilane, 3-Methacryloxypropyltriethoxysilane, 3-Acryloxypropyltrimethoxysilane, 2-Methacryloxyethyltriethoxysilane, 2-Acryloxyethyltriethoxysilane, 3-Methacryloxypropyltris(methoxyethoxy)silane, 3-Methacryloxypropyltris(butoxyethoxy)silane, 3-Methacryloxypropyltris(propoxy)silane, 3-Methacryloxypropyltris(butoxy)silane, 3-Acryloxypropyltris(methoxyethoxy)silane, 3-Acryloxypropyltris(butoxyethoxy)silane, 3-Acryloxypropyltris(butoxy)silane, Vinyltrimethoxysilane, Vinyltriethoxysilane, Vinylethyldichlorsilane, Vinylmethyldiacetoxysilane, Vinylmethyldichlorsilane, Vinylmethyldiethoxysilane, Vinyltriacetoxysilane, Vinyltrichlorsilane, Phenylvinyldiethoxysilane, or Phenylallyldichlorsilane. Furthermore, a modification with a monofunctional silane, an alkylsilane or arylsilane, can be carried out. This has only one functional group, which can covalently bond to the surface of the effect pigment (i.e., to the outermost metal oxide-comprising layer) or, if not completely covered, to the metal surface. The hydrocarbon residue of the silane points away from the effect pigment. Depending on the type and nature of the hydrocarbon residue of the silane, a different degree of hydrophobicity of the effect pigment is achieved. Examples of such silanes are hexadecyltrimethoxysilane, propyltrimethoxysilane, etc. Particularly preferred are effect pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

It may be preferred that a silane having at least one basic group is further used in the sol-gel process.

In addition, or as an alternative to the subsequent modification, adhesion and/or abrasion resistance of effect pigments based on substrate platelets to/in a material, preferably keratinous material, can be significantly increased by additionally using silanes with at least one basic group in the production of the outermost layer, depending on the structure of layer A, B or C. The silanes can also be used in the production of the outer layer. This is particularly advantageous if layer A or layer C are the outermost layer and comprise silica as the metal oxide (hydrate).

In this embodiment of the present disclosure, the sol-gel process for producing layers A or C comprises dispersing the uncoated substrate platelets or substrate platelets already coated with layers A and B, the coloring compound selected from the group of pigments and a silane having at least one basic group in a solution of a metal alkoxide. The silanes are preferably silanes with one, two or three silicon atoms, with one or more hydroxyl groups or hydrolysable groups per molecule and with at least one basic group.

The basic group can be, for example, an amino group, an alkylamino group or a dialkylamino group, which is preferably connected to a silicon atom via a linker.

The silane having at least one basic group is preferably selected from the group of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-Dimethylaminoethyl)triethoxysilane.
(2-Dimethylaminoethyl)trimethoxysilane
1-(2-Dimethylaminoethyl)silantriol
   and mixtures thereof.

Preferably, (3-aminopropyl)triethoxysilane and/or (3-aminopropyl)trimethoxysilane are used.

The metal alkoxide used in the sol-gel process is preferably selected from the group of tetramethyl orthosilicate, tetraethyl orthosilicate, tetraisopropyl orthosilicate, and mixtures thereof. Preferably, tetraethyl orthosilicate is used. Alternatively, or in addition to the tetraalkoxysilane, alkyltrialkoxysilanes can be used in the sol-gel process to produce layers A and/or C. Particularly preferably, alkyltrialkoxysilanes may be used in addition or as an alternative to a tetraalkoxysilane in the sol-gel process to produce a layer comprising (i) silica as a metal oxide and/or metal oxide hydrate and (ii) a coloring compound selected from the group of pigments.

Suitable alkyltrialkoxysilanes include, for example, methyltrimethoxysilane, methyltriethoxysilane, ethyltrimethoxysilane, ethyltriethoxysilane, hexyltrimethoxysilane, hexyltriethoxysilane, octyltrimethoxysilane, octyltriethoxysilane, dodecyltrimethoxysilane, dodecyltriethoxysilane, octyldecyltrimethoxysilane and/or Octyldecyltriethoxysilane.

### Example

First, 200 g Al platelets in the form of VMPs (thickness between 20 nm and 30 nm, d₅₀=12 μm) were suspended in isopropanol. To this mixture, 46 g of tetraethoxysilane and 1 g of Blue 15 pigment (C.I. 74160, D₉₉=20 nm) was added, and the resulting mixture was heated to 60° C. Subsequently, 100 g of water was added followed by 6 g of ammonia and the obtained mixture was stirred for another 4 h. The mixture is then filtered through a glass frit and the filter cake obtained is dried at 120° C. for 12 h. The filter cake is then removed. The colored silica layer accounts for about 40% by weight, based on the total weight of the effect pigment.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. An effect pigment comprising a) a substrate platelet and b) a coating,
wherein the b) coating comprises at least one layer which comprises
(i) a metal oxide and/or metal oxide hydrate; and
(ii) a coloring compound chosen from the group of pigments.

2. Effect pigment according to claim 1, wherein the b) coating completely envelops the substrate platelet.

3. Effect pigment according to claim 1, wherein the b) coating comprises a layer.

4. Effect pigment according to claim 1, wherein the b) coating has a total of at least two layers.

5. Effect pigment according to claim 1, wherein the (i) metal oxide and/or metal oxide hydrate is chosen from silicon (di)oxide, silicon oxide hydrate, aluminum oxide, aluminum oxide hydrate, boron oxide, germanium oxide, manganese oxide, magnesium oxide, iron oxide, cobalt oxide, chromium oxide, titanium dioxide, vanadium oxide, zirconium oxide, tin oxide, zinc oxide, and mixtures thereof.

6. Effect pigment according to claim 1, wherein the (i) metal oxide and/or metal oxide hydrate is silicon dioxide.

7. Effect pigment according to claim 1, wherein the substrate platelet comprises a metal or alloy.

8. Effect pigment according to claim 1, wherein the at least one layer is applied wet-chemically.

9. Effect pigment according to claim 1, wherein a monofunctional or bifunctional organic compound is bound to the coating.

10. Effect pigment according to claim 1, wherein the particle size D₉₀ of the coloring compound is smaller than the layer thickness of the at least one layer.

11. A process for the preparation of an effect pigment comprising a) a substrate platelet and b) a coating, wherein the coating comprises at least one layer which comprises
(i) a metal oxide and/or metal oxide hydrate and
(ii) a coloring compound chosen from pigments, said process comprising the steps of:
(α) suspending the substrate platelet in an organic or aqueous solvent, and
(β) coating the substrate platelet suspended in step (α) with the at least one layer using a sol-gel process.

12. The process according to claim 11, wherein the metal alkoxide and the coloring compound are used in the sol-gel process.

13. The process of claim 12, wherein the metal alkoxide used in the sol-gel process is chosen from tetramethyl orthosilicate, tetraethyl orthosilicate, tetraisopropyl orthosilicate, and mixtures thereof.

14. The process according to claim 11, wherein a silane having at least one basic group is used in the sol-gel process.

15. The process according to claim 11, wherein the substrate platelet used in step (α) is coated with at least one layer of a metal oxide and/or metal oxide hydrate prior to step (α).

16. A process for the preparation of an effect pigment comprising:
suspending Al platelets having a thickness of from about 20 to about 30 nm in isopropanol;
adding tetraethoxysilane and a pigment to the Al platelets suspended in the isopropanol to form a mixture;
heating the mixture; and
filtering the mixture to provide the effect pigment comprising a pigmented silica layer disposed thereon.

17. The process of claim 16 wherein the Al platelets are in the form of vacuum metallized pigments.

18. The process of claim 17 wherein the vacuum metallized pigments have a d₅₀ of 12 μm, the pigment is Blue 15 pigment, and the pigmented silica layer comprises about 40% by weight of the effect pigment.

19. The effect pigment of claim 1 wherein the substrate platelet is an Al platelet having a thickness of from about 20 to about 30 nm, and the metal oxide is silica.

20. The effect pigment of claim 19 wherein the Al platelet is a vacuum metallized pigment that has a d₅₀ of 12 μm, the pigment is Blue 15 pigment, and the coating comprises about 40% by weight of the effect pigment.
